(19) United States
(12) Patent Application Publication
(10) Pub. No.: US 2026/0158023 A1
(43) Pub. Date: Jun. 11, 2026

(54) APOMORPHINE FOR TREATING OR IMPROVING POST-TRAUMATIC STRESS DISORDER

(71) Applicant: POSTECH Research and Business Development Foundation, Pohang-si (KR)

(72) Inventors: Joung-Hun KIM, Pohang-si (KR); Eojin NAM, Pohang-si (KR); Jung Hyun PYO, Pohang-si (KR); Su-Jin NOH, Namyangju-si (KR)

(73) Assignee: POSTECH Research and Business Development Foundation, Pohang-si (KR)

(21) Appl. No.: 19/179,003

(22) Filed: Apr. 15, 2025

(30) Foreign Application Priority Data
Apr. 17, 2024 (KR) 10-2024-0051098
May 20, 2024 (KR) 10-2024-0065455

(51) Int. Cl. A61K 31/473; A01K 67/027; A61P 25/00

(52) U.S. Cl. CPC A61K 31/473; A01K 67/027; A61P 25/00; A01K 2207/35; A01K 2267/035

(57) ABSTRACT

The present invention relates to a pharmaceutical composition for preventing or treating mental illness, comprising apomorphine or a pharmaceutically acceptable salt thereof as an active ingredient, a health functional food composition for preventing or improving mental illness, a method for preventing or treating mental illness, a method for preparing an animal model of induced mental illness, and an animal model prepared according to the method. The composition comprising apomorphine as an active ingredient of the present invention can induce changes in neuroplasticity by specifically enhancing the action of Drd4 dopamine receptors that mediate the formation of fear memories, and thus has the advantage of effectively preventing, treating, or alleviating post-traumatic stress disorder.

### CROSS-REFERENCE TO RELATED APPLICATION

This application claims priority to and the benefit of Korean Patent Application No. 10-2024-0051098, filed on Apr. 17, 2024 and Korean Patent Application No. 10-2024-0065455, filed on May 20, 2024, the disclosure of which is incorporated herein by reference in its entirety.

### BACKGROUND

### 1. Field of the Invention

The present invention relates to apomorphine for treating or alleviating post-traumatic stress disorder, and more specifically, to a pharmaceutical composition for preventing or treating mental illness, a health functional food composition for preventing or improving mental illness, comprising apomorphine or a pharmaceutically acceptable salt thereof as an active ingredient, a method for preventing or treating mental illness, a method for preparing an animal model of induced mental illness, and an animal model prepared according to the method.

### 2. Discussion of Related Art

Post-traumatic stress disorder (PTSD) is a mental disorder that occurs when a person experiences severe events such as war, torture, natural disasters, or accidents, and continues to suffer from distressing re-experiences of these events, thereby expending energy to avoid them. PTSD significantly impacts normal social functioning.

Current pharmacological treatments for PTSD are primarily symptomatic, aimed at relieving symptoms rather than addressing the underlying causes of the disorder. Selective serotonin reuptake inhibitors (SSRIs), initially used as antidepressants, are also prescribed for PTSD. However, they only provide temporary symptom relief without curative effects. Research indicates that 45-60% of patients do not respond to SSRIs for PTSD treatment (Koek et al., Progress in Neuro-Psychopharmacology and Biological Psychiatry, 2016). Additionally, benzodiazepine-based medications used for treating anxiety and insomnia are prescribed for PTSD but have significant side effects, including the reinforcement of traumatic memories, increased risk of suicide, tolerance, and dependency. These medications may even exacerbate the risk of developing PTSD in trauma-exposed patients (Guina et al., Journal of Psychiatric Practice, 2015). Overall, the demand for effective PTSD treatments remains high, but no definitive treatment has been established.

Furthermore, monotherapy with existing drugs often fails to adequately address major symptoms such as sleep disturbances, with frequent relapses and the necessity for continuous medication to prevent recurrence. Non-pharmacological treatments heavily depend on patient motivation and require prolonged treatment periods, often resulting in only partial symptom relief, thus failing to establish a robust therapeutic approach for PTSD.

Additionally, conventional PTSD treatments focus on symptomatic relief through anti-anxiety and antidepressant medications rather than targeting the underlying pathophysiology of the disorder. Due to the psychological and physiological side effects associated with these medications, they are not suitable for long-term use. Therefore, there is a need for therapeutic approaches that address the underlying mechanisms of PTSD.

Fear memories are stored within the amygdala, where strong neural circuitry connections result in heightened fear responses, whereas weak connections result in reduced fear responses. It has been demonstrated that Drd4 agonists can directly attenuate fear responses (Kwon et al., Neuron, 2015). Npas4, an Immediate Early Gene (IEG), is known to play a critical role in the formation of fear memories in response to traumatic stimuli within the amygdala. Stress-induced expression of Npas4 in neurons within the amygdala induces neural plasticity through the downstream signaling of Drd4, contributing to the formation of fear memories. Enhancing the action of Drd4 or its downstream signaling mediated by Npas4-expressing neurons has been suggested as a potential strategy to alleviate or treat PTSD symptoms (Ko et al., Cell Reports, 2023).

Apomorphine (APO), with the chemical formula C₁₇H₁₇NO₂, is a compound derived from morphine through a reaction with mineral acids. It is an FDA-approved drug commonly used in the treatment of Parkinson's disease by activating dopamine D2-like receptors. Structurally similar to dopamine, apomorphine acts by stimulating dopamine autoreceptors located in dopaminergic neurons. Through this mechanism, it activates dopamine receptors in the nigrostriatal pathway, limbic system, hypothalamus, and pituitary gland, resulting in improvements in motor function and other therapeutic effects.

Accordingly, the inventors of the present invention confirmed the therapeutic effect of apomorphine on post-traumatic stress disorder (PTSD), a type of mental illness, to treat post-traumatic stress disorder (PTSD), thereby completing the present invention.

### SUMMARY OF THE INVENTION

The purpose of the present invention is to provide a pharmaceutical composition for preventing or treating mental illness, comprising apomorphine or a pharmaceutically acceptable salt thereof as an active ingredient.

Another purpose of the present invention is to provide a health functional food composition for preventing or alleviating mental illness, comprising apomorphine or a pharmaceutically acceptable salt thereof as an active ingredient.

Another purpose of the present invention is to provide a method for preventing or treating mental illness, comprising administering apomorphine to a subject.

Another purpose of the present invention is to provide a method for preparing an animal model of induced mental illness, comprising restricting movement of animal for applying restraint stress; and applying sound and electrical stimuli (fear stimuli) and sound-only (safety stimuli) to the animal, respectively.

Another purpose of the present invention is to provide an animal model prepared according to the method.

In order to achieve the purpose, the present invention provides a pharmaceutical composition for preventing or treating mental illness, comprising apomorphine or a pharmaceutically acceptable salt thereof as an active ingredient.

In addition, the present invention provides a health functional food composition for preventing or alleviating mental illness, comprising apomorphine or a pharmaceutically acceptable salt thereof as an active ingredient.

In addition, the present invention provides a method for preventing or treating mental illness, comprising administering apomorphine to a subject.

In addition, the present invention A method for preparing an animal model of induced mental illness, comprising restricting movement of animal for applying restraint stress; and applying sound and electrical stimuli (fear stimuli) and sound-only (safety stimuli) to the animal, respectively.

In addition, the present invention provides an animal model prepared according to the method.

The present invention provides a composition comprising apomorphine as an active ingredient, and thus can be effectively utilized for the prevention and treatment of mental illness. In particular, the composition of the present invention comprising apomorphine as an active ingredient can induce changes in neuroplasticity by specifically enhancing the action of Drd4 dopamine receptors that mediate the formation of fear memories, and thus has an advantage in that it can effectively prevent, treat, or improve post-traumatic stress disorder. In particular, a low concentration of apomorphine according to an embodiment of the present invention has an excellent advantage in that it can improve symptoms of post-traumatic stress disorder, which was difficult to treat in the past, by more effectively inhibiting the pathogenesis of post-traumatic stress disorder.

### BRIEF DESCRIPTION OF THE DRAWINGS

FIG. 1 shows the process of preparing an animal model of post-traumatic stress disorder by inducing restraint stress.

FIG. 2 shows the results of confirming whether the animal model of post-traumatic stress disorder by inducing restraint stress has a stress disorder pattern.

FIG. 3 shows the effect of alleviating the stimulus identification of fear response in a post-traumatic stress model according to the concentration of apomorphine treatment.

FIG. 4 shows the process of preparing an animal model of post-traumatic stress disorder by inducing one-time persistent stress.

FIG. 5 shows the results of confirming whether the animal model of post-traumatic stress disorder by inducing one-time persistent stress has a stress disorder pattern.

FIG. 6 shows the results of confirming whether apomorphine reduces anxiety in a one-time persistent stress model through the open field test and the elevated plus maze test.

FIG. 7 shows the results of confirming whether the one-time persistent stress model has an improvement in stimulus identification fear according to the concentration of apomorphine treatment.

FIG. 8 shows the results of examining whether stimulus identification fear is alleviated by apomorphine treatment in a one-time persistent stress model induced from mice lacking the drd4 receptor and normal mice.

FIG. 9 shows the results of examining whether stimulus identification fear is improved according to the concentration of paroxetine treatment in a one-time persistent stress model.

FIG. 10 shows the results of examining whether stimulus identification fear is improved according to the concentration of paroxetine treatment in a one-time persistent stress model.

FIG. 11 shows the results of performing active avoidance response in a one-time persistent stress-induced post-traumatic stress disorder model.

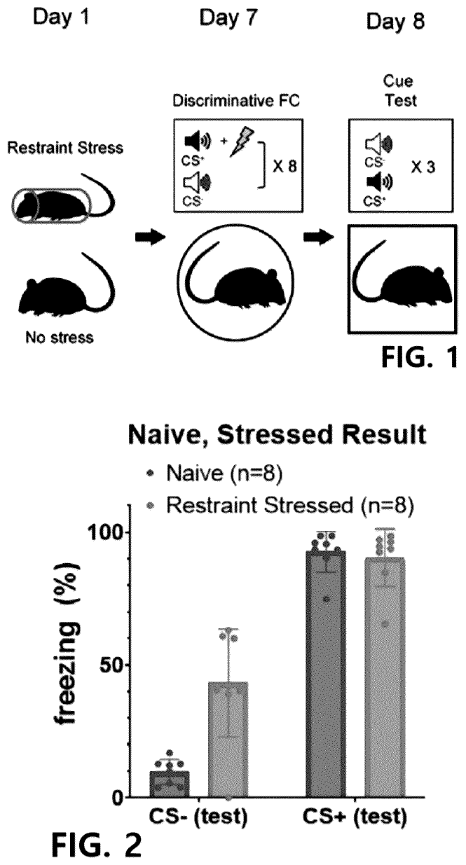

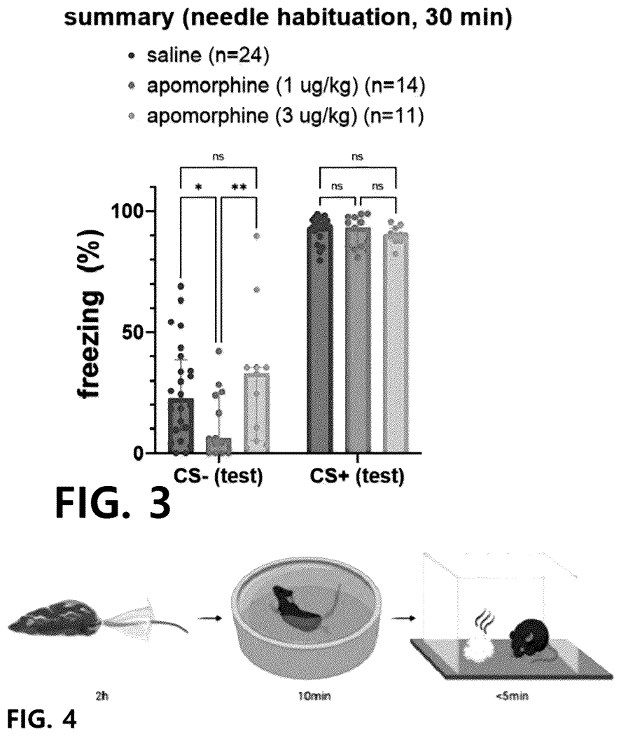

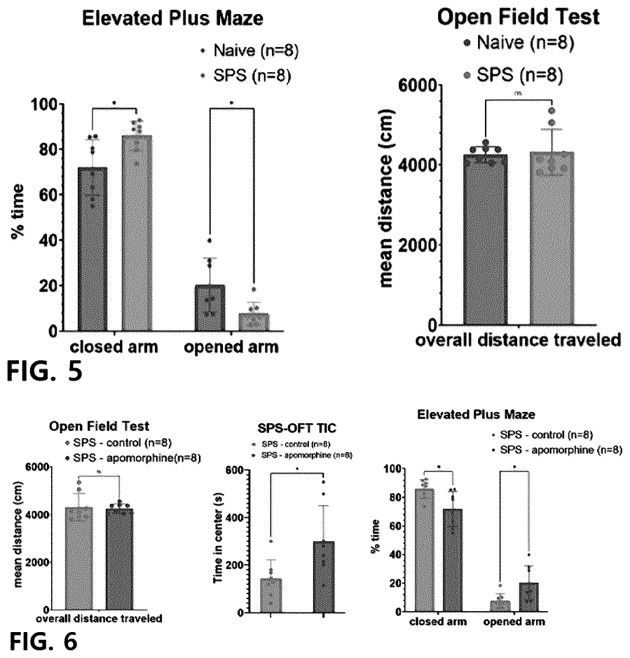

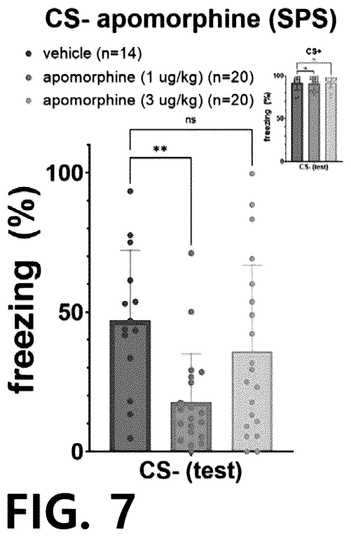

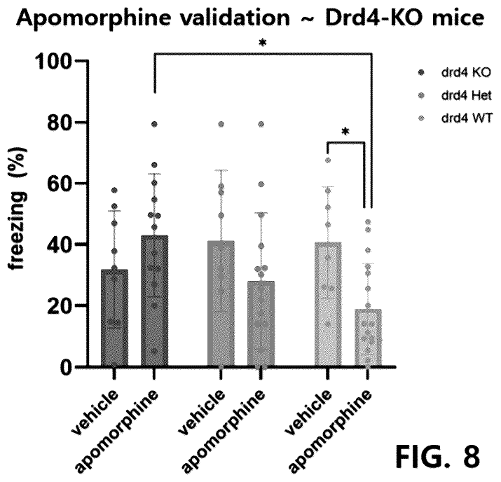

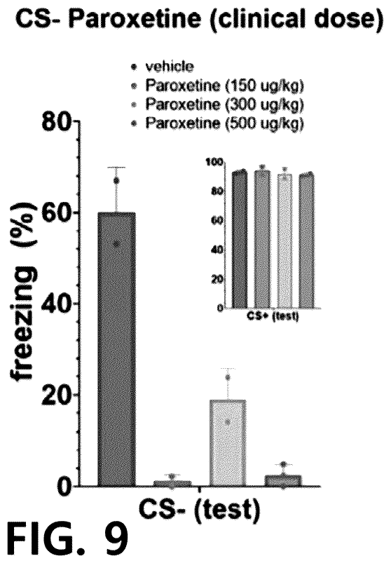

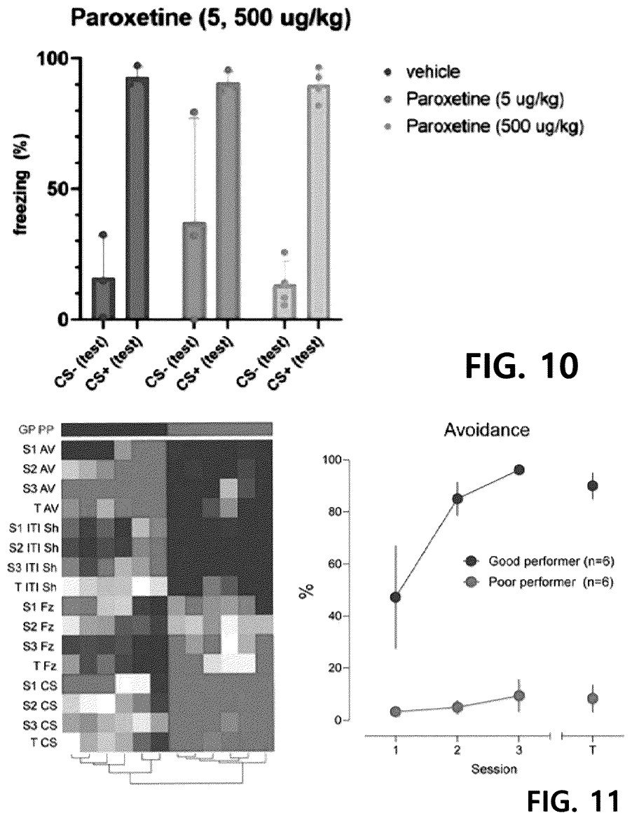

### DETAILED DESCRIPTION OF EXEMPLARY EMBODIMENTS

In the case of neuropsychiatric drugs, side effects inevitably occur when the target expression is high or widely located. Currently, drugs being studied with the goal of clinical entry include endocannabinoid receptor inhibitors and acetylcholine receptor inhibitors, but there are concerns about side effects due to the wide range of targets and high expression. Dopamine receptor type 4 (Drd4) is limited to the amygdala, which is the center of the fear circuit, and not only is there no concern about side effects due to low expression, but it is also excellent in treating PTSD. Accordingly, the present invention solves the problem of the side effects by utilizing apomorphine, and solves the problem of causal treatment by directly controlling fear memory by activating Drd4.

Hereinafter, the present invention will be described in more detail.

The present invention provides a pharmaceutical composition for preventing or treating mental illness, comprising apomorphine or a pharmaceutically acceptable salt thereof as an active ingredient.

In the present invention, apomorphine may be an agonist for dopamine receptor type 4 (Drd4).

In the present invention, dopamine is a neurotransmitter essential for neural signal transmission found in the brain of animals including humans, and the dopamine receptor is a seven-transmembrane (G protein-coupled) peptide that transmits a binding signal with dopamine into cells.

In the present invention, apomorphine may activate dopamine receptor type 4 (Drd4). According to one embodiment of the present invention, apomorphine was used in the present invention to enhance Drd4 signaling. Apomorphine acts as a nonspecific dopamine agonist at high concentrations, but acts as a Drd4-specific dopamine agonist at low concentrations. However, since dopamine agonists often have side effects or cannot penetrate the blood-brain barrier, their use is limited. Therefore, in the present invention, it was confirmed that post-traumatic stress disorder (PTSD) can be treated by selectively activating Drd4 using a low concentration of apomorphine that can penetrate the blood-brain barrier.

In the present invention, the composition may suppress fear response or suppress reconsolidation and retrieval of fear memory.

In the present invention, the concentration of apomorphine in the composition may be 0.1 ug/kg to 2.5 ug/kg, preferably, the concentration of apomorphine in the composition may be 0.5 ug/kg to 1.5 ug/kg, more preferably, the concentration of apomorphine in the composition may be 1 ug/kg.

In the present invention, preventing may mean any act of suppressing or delaying mental illness by administering the composition of the present invention to a subject.

In the present invention, treating or alleviating may mean any act of improving or benefiting the symptoms of mental illness by administering the composition of the present invention to a subject.

In the present invention, the pharmaceutical composition may further comprise components such as existing therapeutic active ingredients, other adjuvants, and pharmaceutically acceptable carriers.

In the present invention, the pharmaceutically acceptable carrier refers to a carrier or diluent that does not stimulate a living organism and does not inhibit the biological activity and properties of the administered compound. In a composition formulated as a liquid solution, acceptable pharmaceutical carriers are sterile and biocompatible, and include saline solution, sterile water, Ringer's solution, buffered saline, albumin injection solution, dextrose solution, maltodextrin solution, glycerol, ethanol, and one or more of these components can be mixed and used. Additionally, antioxidants, buffers, bacteriostatic agents, and other common additives can be added as needed. In addition, diluents, dispersants, surfactants, binders, and lubricants can be additionally added to formulate the composition into injectable formulations such as aqueous solutions, suspensions, and emulsions, pills, capsules, granules, or tablets.

In the present invention, the pharmaceutical composition may be in various oral or parenteral dosage forms. When formulated, it is prepared using diluents such as fillers, bulking agents, binders, wetting agents, disintegrants, and surfactants or excipients that are commonly used. Solid preparations for oral administration include tablets, pills, powders, granules, and capsules, and these solid preparations are prepared by mixing one or more compounds with at least one excipient, such as starch, calcium carbonate, sucrose or lactose, gelatin, and the like. In addition to simple excipients, lubricants such as magnesium stearate and talc are also used. Liquid preparations for oral administration include suspensions, oral solutions, emulsions, and syrups, and in addition to commonly used simple diluents such as water and liquid paraffin, various excipients such as wetting agents, sweeteners, fragrances, and preservatives may be included. Preparations for parenteral administration include sterile aqueous solutions, non-aqueous solutions, suspensions, emulsions, lyophilized preparations, and suppositories. Non-aqueous solutions and suspensions may include propylene glycol, polyethylene glycol, vegetable oils such as olive oil, and injectable esters such as ethyl oleate. Suppository bases may include witepsol, macrogol, Tween 61, cacao butter, laurin butter, glycerogelatin, and the like.

In the present invention, the pharmaceutical composition may be administered in a pharmaceutically effective amount. The pharmaceutically effective amount means an amount sufficient to treat a disease at a reasonable benefit/risk ratio applicable to medical treatment, and the effective dosage level may be determined based on the type and severity of the individual, age, sex, activity of the drug, sensitivity to the drug, administration time, administration route, and excretion rate, treatment period, concurrently used drugs, and other factors well known in the medical field. The composition of the present invention may be administered as an individual therapeutic agent or in combination with other therapeutic agents, and may be administered sequentially or simultaneously with conventional therapeutic agents. And may be administered singly or in multiple doses. It is important to administer an amount that can achieve the maximum effect with the minimum amount without side effects by considering all of the above factors, and this can be easily determined by those skilled in the art.

In the present invention, mental illness refers to a pathological mental state that affects a person's thoughts, emotions, and behaviors, and collectively refers to a state in which mental functions are impaired. The mental illness includes, but is not limited to, post-traumatic stress disorder (PTSD), panic disorder, autism spectrum disorders, schizophrenia, depression, anxiety disorders, and attention deficit/hyperactivity disorder (ADHD). However, it is preferable that the mental illness is post-traumatic stress disorder (PTSD).

In the present invention, post-traumatic stress disorder (PTSD) refers to a mental illness that can occur after receiving mental trauma through a severe accident, and has hypersensitivity, re-experiencing shock, or emotional avoidance or paralysis as its main symptoms.

In addition, the present invention provides a health functional food composition for preventing or alleviating mental illness, comprising apomorphine or a pharmaceutically acceptable salt thereof as an active ingredient.

The characteristics corresponding to the apomorphine described above included in the health functional food composition can be replaced in the above-described part, so their description is omitted.

In the present invention, the health functional food composition can be manufactured as a specific health food, a food with high medical and medical effects processed to efficiently exhibit a bioregulatory function in addition to providing nutrition, and the food can be mixed with a functional food, a health food, or a health supplement food, depending on the case, and can be manufactured in various forms such as a tablet, capsule, powder, granule, liquid, or pill to obtain a useful effect.

In the present invention, the health functional food composition may comprise additional ingredients that are commonly used in food compositions and can improve smell, taste, sight, etc. For example, it can include vitamins A, C, D, E, B1, B2, B6, B12, niacin, biotin, folate, pantothenic acid, etc. It may also contain minerals such as zinc (Zn), iron (Fe), calcium (Ca), chromium (Cr), magnesium (Mg), manganese (Mn), and copper (Cu). It may also contain amino acids such as lysine, tryptophan, cysteine, and valine. In addition, food additives such as preservatives (potassium sorbate, sodium benzoate, salicylic acid, sodium dehydroacetate, etc.), bactericides (bleaching powder and high-purity bleaching powder, sodium hypochlorite, etc.), antioxidants (butylated hydroxyanisole (BHA), butylated hydroxy toluene (BHT), etc.), coloring agents (tar color, etc.), color developer (sodium nitrite, sodium nitrite, etc.), bleaching agents (sodium sulfite), seasonings (MSG, monosodium glutamate, etc.), sweeteners (dulcin, cyclamate, saccharin, sodium, etc.), flavorings (vanillin, lactones, etc.), leavening agents (alum, D-potassium hydrogen tartrate, etc.), reinforcing agents, emulsifiers, thickeners (glutinating agents), film agents, gum bases, foam suppressants, solvents, and improvers can be added. The above additives can be selected depending on the type of food and used in an appropriate amount.

In the present invention, when the health functional food composition is used as a food additive, it can be added as it is or used together with other foods or food ingredients, and can be used appropriately according to a conventional method.

In addition, the present invention provides a method for preventing or treating mental illness, comprising administering apomorphine to a subject.

The characteristics corresponding to the apomorphine described above included in the method can be replaced in the above-described part, so their description is omitted.

In the present invention, the subject may mean all animals including humans who have developed or are likely to develop mental illness. The animal may be a mammal such as a cow, horse, sheep, pig, goat, camel, antelope, dog, cat, etc. that requires treatment for symptoms similar to humans, but is not limited thereto.

In the present invention, the subject may have damaged or deficient dopamine receptor type D4.

In the present invention, the apomorphine may be an agonist for dopamine receptor type 4 (Drd4).

In the present invention, the apomorphine may activate dopamine receptor type 4 (Drd4).

In the present invention, the concentration of the apomorphine may be 0.1 ug/kg to 2.5 ug/kg, preferably, the concentration of apomorphine may be 0.5 ug/kg to 1.5 ug/kg, more preferably, the concentration of apomorphine may be 1 ug/kg.

In the present invention, there is no limitation on the administration method as long as it can reach the target tissue. For example, the administration may be at least one selected from the group consisting of intraperitoneal administration, oral administration, inhalation administration, intravenous administration, intramuscular administration, subcutaneous administration, dermal administration, and rectal administration. In addition, it is preferable to administer once or several times a day.

In the present invention, the mental illness may be at least one selected from the group consisting of post-traumatic stress disorder (PTSD), panic disorder, autism spectrum disorders, schizophrenia, depression, anxiety disorders, and attention deficit/hyperactivity disorder (ADHD).

In addition, the present invention provides a method for preparing an animal model of induced mental illness, comprising restricting movement of the animal for applying restraint stress; and applying sound and electrical stimuli (fear stimulation) and sound-only (safety stimuli) to the animal, respectively.

The characteristics corresponding to the apomorphine described above included in the method for preparing the animal model can be replaced in the above-described part, and therefore, the description thereof is omitted.

In the present invention, method may further comprise conducting a forced swim and ether anesthesia after the applying.

In the present invention, the mental illness may be at least one selected from the group consisting of post-traumatic stress disorder (PTSD), panic disorder, autism spectrum disorders, schizophrenia, depression, anxiety disorders, and attention deficit/hyperactivity disorder (ADHD).

In addition, the present invention provides an animal model prepared according to the method.

Hereinafter, in order to help understand the present invention, examples will be given and described in detail. However, the following examples are only intended to illustrate the contents of the present invention, and the scope of the present invention is not limited to the following examples. The examples of the present invention are provided to more completely explain the present invention to a person having average knowledge in the art.

### Example 1

### Therapeutic Effect of Apomorphine in a Model of Post-Traumatic Stress Disorder Induced by Restraint Stress

### 1.1. Preparation of an Animal Model of Post-traumatic Stress Disorder Induced by Restraint Stress

In order to observe the therapeutic effect of apomorphine on post-traumatic stress disorder, an animal model that modeled post-traumatic stress disorder was prepared.

C57BL/6 mice were stressed by restricting movement for 2 hours (restraint stress), and one week later, two sounds were simultaneously applied: one with sound-only (safety stimuli) and the other with sound paired with electrical stimulation (fear stimuli). The next day, generalization, the freezing level to the safety stimulus (CS-), which is one of the indicators of post-traumatic stress disorder, was measured through fear responses to the two sounds (stimulus identification fear conditioning experiment).

Compared to non-stressed mice, stressed mice showed a higher fear response to the safety stimulus, confirming that this model can model post-traumatic stress disorder.

### 1.2. Confirmation of the Effect of Apomorphine on Alleviating Stimulus Identification Fear Response in the Restraint Stress Model

To confirm the effect of apomorphine on the post-traumatic stress model according to concentration, apomorphine of different concentrations was administered 30 minutes before the fear test.

It was confirmed that CS-freezing did not change in apomorphine (3 ug/kg), but CS-freezing decreased in apomorphine (1 ug/kg). This is a phenomenon that occurs because apomorphine is not a drug that acts only on Drd4, but acts on all dopamine receptors. However, since it has high affinity for Drd4, it is presumed that it will react specifically to Drd4 at low concentrations. Through this, it is presumed that apomorphine at 1 ug/kg acts as an agonist that reacts specifically to Drd4, thereby reducing the fear response to CS- in the stimulus identification fear conditioning experiment. Therefore, in the present invention, it was confirmed that apomorphine at 1 ug/kg is effective for post-traumatic stress disorder.

### Example 2

### Therapeutic Effect of Apomorphine in a Post-traumatic Stress Disorder Model Induced by One-time Persistent Stress

### 2.1. Preparation of an Animal Model of Post-Traumatic Stress Disorder Induced by One-Time Persistent Stress

In order to strengthen the fear response to safety stimuli, a one-time persistent stress was introduced to apply stronger stress. The one-time persistent stress consists of the existing 2-hour restraint stress, 10-minute forced swim test, and 5-minute ether anesthesia.

As a result, it was confirmed that when one-time persistent stress was applied, the time spent in the open arm significantly decreased in the elevated plus maze test, which is one of the tests for anxiety disorders. However, since there was no difference in the mean distance in the open field test, it was found that it did not affect other walking functions.

### 2.2. Confirmation of the Anxiety-reducing Effect of Apomorphine in the One-time Persistent Stress Model

Since the post-traumatic stress model shows general anxiety disorder, it was confirmed whether apomorphine reduces anxiety symptoms.

The level of anxiety symptoms can be determined through the time spent in the center in the open field test. In the post-traumatic stress model, the time spent in the center decreased, but it was found that the time spent in the center significantly increased after apomorphine administration. However, since the mean distance showed no difference, it was found that the overall walking function was not affected. In addition, in the post-traumatic stress model, the time spent in the open arm in the elevated plus maze test decreased, but it was confirmed that it significantly increased after apomorphine administration. Through this, it was found that apomorphine is effective in alleviating anxiety symptoms in the post-traumatic stress model.

### 2.3. Confirmation of the Effect of Apomorphine on Alleviating Stimulus Identification Fear in a One-Time Persistent Stress Model

We observed the fear response in a stimulus identification fear conditioning experiment when apomorphine (1 ug/kg, 3 ug/kg) was administered one week after a one-time persistent stress, which is a more powerful post-traumatic stress model.

We confirmed that CS-freezing did not change in apomorphine (3 ug/kg), but CS-freezing decreased in apomorphine (1 ug/kg). Through this, we were able to find out that apomorphine is effective in reducing fear response to safety stimuli in another post-traumatic stress model.

2.4. Confirmation of the Effect of Apomorphine on Alleviating Stimulus Identification Fear and its Relationship with drd4

To confirm whether the above effect of apomorphine on alleviating stimulus identification fear is a result of its specific action on drd4, a stimulus identification fear conditioning experiment was conducted in the same manner as in Example 2.3 using normal mice born from the same parents as mice lacking drd4 receptors.

As in Example 2.3, it was confirmed that the fear response of normal mice (drd4 WT) decreased after apomorphine administration compared to vehicle administration, but in mice lacking drd4 receptors (drd4 KO), there was no difference in the fear response between apomorphine administration and vehicle administration, indicating that the effect was derived from its specific action on drd4.

### Example 3

### Therapeutic Effects of Existing Treatments in a Model of Post-Traumatic Stress Disorder Induced by One-time Persistent Stress

### 3.1. Confirmation of the Effect of Paroxetine on Alleviating Stimulus Identification Fear in a Model of One-Time Persistent Stress

In order to compare the effect of apomorphine with that of the existing treatment, paroxetine, a stimulus identification fear conditioning experiment was conducted after stress was applied under the same conditions.

Since the clinical dose of paroxetine is 10 mg and 20 mg, assuming an adult body weight of 60 kg, it is 0.1666 mg/kg and 0.3333 mg/kg, but considering that the bioavailability is low when administered orally, it was determined to be approximately 150 ug/kg and 300 ug/kg and administered. In order to confirm the difference at a higher concentration, the effect at 500 ug/kg was also compared. As a result, it was confirmed that the fear response to safety stimuli was reduced at all concentrations in the stimulus identification fear conditioning experiment.

### 3.2. Confirmation of the Effect According to Paroxetine Concentration on Alleviating Stimulus Identification Fear in a One-Time Persistent Stress Model

In Example 3.1, an experiment was conducted on the clinical dose of paroxetine, but it was confirmed that apomorphine was effective at 1 ug/kg, so there was a difference of more than 150 times compared to paroxetine. Therefore, the effect at a low concentration was compared. As a result, through the stimulus identification fear conditioning experiment, there was no significant difference in the fear response to safety stimuli at 5 ug/kg of paroxetine.

### Example 4

### Active Avoidance Response Performance in a Post-Traumatic Stress Disorder Model Induced by One-time Persistent Stress

Active avoidance response was performed in a post-traumatic stress model. In a model that did not induce stress, Good performers who generally performed well were 73%, and Poor performers who did not perform well were 27% (Choi., et al, Learning and Memory, 2010). On the other hand, in one-time persistent stress model, Good performers and Poor performers were 50% each, confirming that the active avoidance response was not easier in a post-traumatic stress model than in a model that did not induce stress.

As the specific parts of the present invention have been described in detail above, it is obvious to those skilled in the art that such specific descriptions are merely preferred embodiments and that the scope of the present invention is not limited thereby. In other words, the substantial scope of the present invention is defined by the appended claims and their equivalents.

What is claimed is:

1. A method for preventing or treating mental illness, comprising administering apomorphine to a subject in need thereof.

2. The method of claim 1, wherein the subject has damaged or deficient dopamine receptor D4.

3. The method of claim 1, wherein the apomorphine is an agonist for dopamine receptor D4.

4. The method of claim 1, wherein the apomorphine activates dopamine receptor D4.

5. The method of claim 1, wherein a concentration of the apomorphine 0.1 ug/kg to 2.5 ug/kg.

6. The method of claim 1, wherein the apomorphine is administered via at least one selected from the group consisting of intraperitoneal administration, oral administration, inhalation administration, intravenous administration, intramuscular administration, subcutaneous administration, intradermal administration, and rectal administration.

7. The method of claim 1, wherein the mental illness includes at least one selected from a group consisting of post-traumatic stress disorder (PTSD), panic disorder, autism spectrum disorders, schizophrenia, depression, anxiety disorders, and attention deficit/hyperactivity disorder (ADHD).

8. A method for preparing an animal model of induced mental illness, comprising:
restricting movement of animal for restraint stress; and
applying sound and electrical stimuli (fear stimuli) and sound-only (safety stimuli) to the animal, respectively.

9. The method of claim 8, wherein the method further comprising conducting a forced swim and ether anesthesia after the applying.

10. The method of claim 8, wherein the mental illness includes at least one selected from a group consisting of post-traumatic stress disorder (PTSD), panic disorder, autism spectrum disorders, schizophrenia, depression, anxiety disorders and attention deficit/hyperactivity disorder (ADHD).

11. An animal model prepared according to the method of claim 8.

12. An animal model prepared according to the method of claim 9.

13. An animal model prepared according to the method of claim 10.

14. A health functional food composition for preventing or alleviating mental illness, comprising apomorphine or a pharmaceutically acceptable salt thereof as an active ingredient.

15. The health functional food composition of claim 12, wherein the mental illness includes at least one selected from a group consisting of post-traumatic stress disorder (PTSD), panic disorder, autism spectrum disorders, schizophrenia, depression, anxiety disorders and attention deficit/hyperactivity disorder (ADHD).
